(19) United States
(12) Patent Application Publication
(10) Pub. No.: US 2024/0139001 A1
(43) Pub. Date: May 2, 2024

(54) PROSTHETIC FEEDBACK DEVICE

(71) Applicant: UNIVERSITÀ DI PISA, Pisa (IT)

(72) Inventors: Federica BARONTINI, Osimo (IT); Antonio BICCHI, Massa (IT); Matteo BIANCHI, Pisa (IT); Manuel Giuseppe CATALANO, Pisa (IT); Giorgio GRIOLI, Pisa (IT); Cristiano PETROCELLI, Pisa (IT); Mattia POGGIANI, Pisa (IT)

(21) Appl. No.: 18/548,586

(22) PCT Filed: Feb. 8, 2022
(86) PCT No.: PCT/IB2022/051104

§ 371 Date: Sep. 1, 2023

(30) Foreign Application Priority Data
Mar. 4, 2021 (IT) 102021000005099

(51) Int. Cl. A61F 2/58; A61F 2/70; A61F 2/68

(52) U.S. Cl. CPC A61F 2/583; A61F 2/70; A61F 2002/6827; A61F 2002/701

(57) ABSTRACT

A prosthetic feedback device to be associated with a prosthesis equipped with a motor for controlling the same prosthesis is provided The device includes an attachment body configured to be in direct contact with the user; at least one air chamber interposed at least partially between the body and the user; an inflation circuit for inflating the chamber; and a circuit board to control the inflation of the chamber by the motor.

The present invention relates to a prosthetic feedback device of the type specified in the preamble to the first claim.

In particular, the present invention relates to a device configured to provide feedback to a prosthesis user from the interaction of the prosthesis with the external environment. More particularly, the device is associable with a limb, preferably an upper limb and more preferably a hand so as to provide feedback of the interaction, suitably tactile, of the hand with an object or other element of the external environment.

Artificial limbs, and in particular prosthetic hands, are currently known to be components designed to partially or completely replace a part of the human body lost due to acquired causes such as a traumatic event or congenital causes, restoring body image and functionality.

The artificial hands are therefore designed to interact with the external environment and, for example, to grip an object.

Currently, many prosthetic hands are under-actuated, i.e. with fewer actuators than the number of degrees of freedom.

An example of an under-actuated artificial hand involves an actuator for each finger connected to the individual phalanges in order to control their rotation. The fingers can thus be moved independently of each other. Another example is that of artificial hands in which a single actuator (at most two) is provided which, connected to the various phalanges, allows simultaneous flexion to be controlled.

Examples of artificial hands are described in U.S. Pat. Nos. 10,912,661B2 and 10,695,913B2. The known technique described includes some important drawbacks.

In particular, these artificial prostheses (such as actuated or under-actuated hands) do not allow the user to have feedback of the interaction between the prosthesis and the external environment such as those related to gripping such as hardness, softness of the gripping object.

This drawback is particularly relevant in the case of loss due to acquired causes, where a latent sensation remains (i.e. an image/processing that remains in the brain related to the sensation one had before the loss) and is no longer obtained by gripping the object.

In this situation, the technical task at the basis of the present invention is to devise a prosthetic feedback device capable of substantially obviating at least part of the aforementioned drawbacks.

In the context of said technical task, it is an important scope of the invention to obtain a prosthetic feedback device and in particular a prosthesis capable of providing feedback of the interaction between the prosthesis and the external environment.

In particular, it is an important scope of the invention to achieve a prosthetic feedback device capable of communicating/making the user perceive one or more characteristics of the taken object such as hardness and softness.

The specified technical task and purposes are achieved by a prosthetic feedback device as claimed in the appended claim 1. Examples of preferred embodiments are described in the dependent claims.

The features and advantages of the invention are hereinafter clarified by the detailed description of preferred embodiments of the invention, with reference to the appended drawings, wherein:

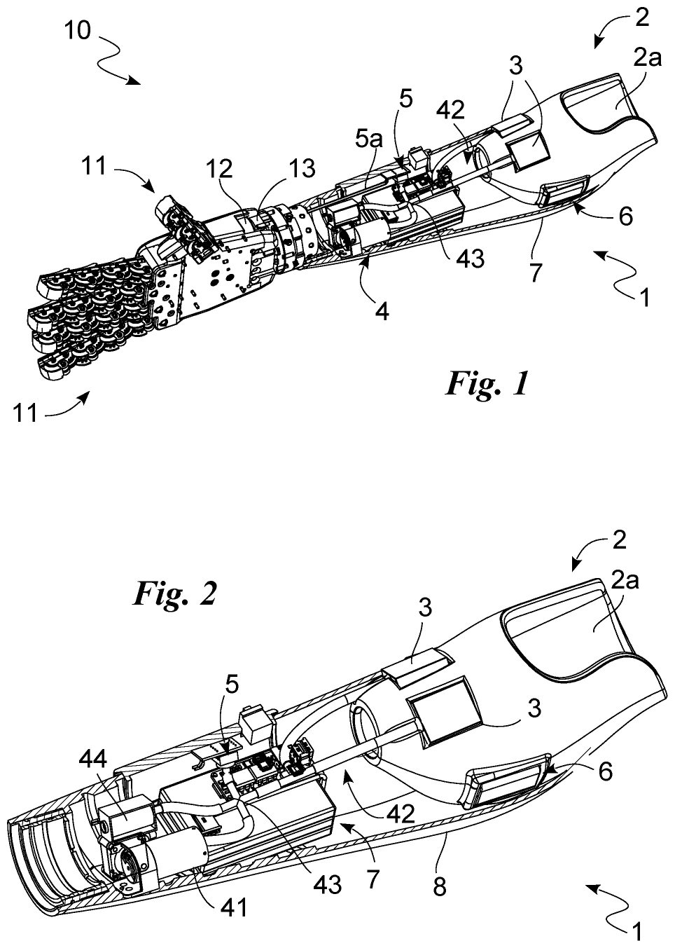

the FIG. 1 illustrates, to scale, a possible application of the prosthetic feedback device; and

the FIG. 2 illustrates, to scale, prosthetic feedback device according to the invention.

In the present document, the measurements, values, shapes and geometric references (such as perpendicularity and parallelism), when associated with words like “about” or other similar terms such as “approximately” or “substantially”, are to be considered as except for measurement errors or inaccuracies due to production and/or manufacturing errors, and, above all, except for a slight divergence from the value, measurements, shape, or geometric reference with which it is associated. For instance, these terms, if associated with a value, preferably indicate a divergence of not more than 10% of the value.

Moreover, when used, terms such as “first”, “second”, “higher”, “lower”, “main” and “secondary” do not necessarily identify an order, a priority of relationship or a relative position, but can simply be used to clearly distinguish between their different components.

The measurements and data reported in this text are to be considered, unless otherwise indicated, as performed in the International Standard Atmosphere ICAO (ISO 2533:1975).

Unless otherwise specified, as results in the following discussions, terms such as “treatment”, “computing”, “determination”, “calculation”, or similar, refer to the action and/or processes of a computer or similar electronic calculation device that manipulates and/or transforms data represented as physical, such as electronic quantities of registers of a computer system and/or memories in, other data similarly represented as physical quantities within computer systems, registers or other storage, transmission or information displaying devices.

With reference to the Figures, the prosthetic feedback device according to the invention is globally referred to as number 1.

The device 1 is configured to be associated with a prosthesis 10 so as to provide feedback on the interaction of the prosthesis 10 with the external environment. Preferably, the device 1 is configured to be associated with an appropriately upper limb and in particular a hand.

The prosthesis 10 may comprise a device 1.

The prosthesis 10 may be a robotic hand preferably under-actuated.

The hand may comprise at least one finger 11 and in particular a plurality of fingers 11. Preferably it comprises five fingers 11.

The prosthesis 10 may comprise at least one motor 12 controlling the prosthesis 10. The at least one motor 12 may be configured to control the hand and for example the assumption of an object grasping pose.

Preferably, the prosthesis 10 (in detail the hand) is under-actuated and therefore the prosthesis 10 may comprise a number of motors 12 that is less than the degrees of freedom of the prosthesis 10. In particular, the prosthesis 10 may comprise only one motor 12 or alternatively two motors 12.

In the case of prosthesis 10 identifiable in a hand, the one motor 12 may control all fingers 11.

The motor 12 may define a motion output through which it controls the movement of the prosthesis 10.

The prosthesis 10 may comprise an encoder 13 measuring the output of the motor 12, or rather the motion output of the motor 12. Preferably the output is a shaft and the encoder measures the number of revolutions and/or the angle of rotation of the shaft or rather the output.

The at least one motor 12 controls the prosthesis 10 (in detail the hand) by defining distinct operating parameters depending on the operation (grip) to be performed.

Some examples of operating parameters of the motor 12 may be operating time, inrush (or initial) current, actual power (i.e. delivered and thus used by the prosthesis 10 to perform the operation) and current drawn by the motor 12 to command the execution of the operation.

Preferably an operating parameter of each motor 12 is the current residual. The residual current identifies the difference between the actual current and the ideal current.

In this document, the expression “real current” is the current absorbed by the prosthesis 10 (in detail by the hand) when performing a real operation and for example interacting with an object (performing grasping of an object); while the expression “ideal current” is the current absorbed by the prosthesis 10 (in detail by the hand) under ideal conditions such as when performing an operation without interacting with an object (for example assuming a grasping pose without grasping an object).

The device 1 may comprise a user attachment body 2.

Body 2 is configured to directly contact the user when prosthesis 1 is in use, i.e. worn by the user. It thus defines a contact surface 2a of the device 1 and thus of the prosthesis 10 with the user.

The binding of the device 1 and thus the prosthesis 10 to the user can be performed at the contact surface 2a.

The contact surface 2a may be concave and configured to accommodate, in use, the amputated extremity of a limb and in particular of an arm or forearm. In detail, the contact surface 2a and to be precise the body 2 may be dome-shaped.

The body 2 may define at least one open section through which to contact the user and for example insert the amputated limb into the prosthesis 10.

The device 1 may comprise at least one air chamber 3 integrated into the body 2 and configured to contact the user appropriately at the contact surface. Suitably it comprises multiple air chambers 3 and preferably two.

The chambers 3 may be mutually spaced and therefore not in contact so as to facilitate discrimination between the chambers by the user.

Each chamber 3 may be interposed at least partially between at least part of the body 2 and the user, and in particular between at least part of the contact surface 2a and the user. It is therefore configured to protrude from the contact surface 2a (in detail at least when inflated) so as to be able to press against the skin of the user by defining a feedback for the user.

The at least one air chamber 3 may be placed proximal to the portion of the contact surface 2a proximal to the part of the user where the skin is less deformable and in particular at the area of a joint where it deforms (in detail moves and/or contracts) during a normal movement of the user. For example, in the case of a prosthesis to be associated with an arm the at least one chamber 3 is located at the contact surface 2a at the inner face of the elbow.

The at least one air chamber 3 may be placed in proximity to the point of greatest gravitational potential of the contact surface 2a when the prosthesis 1 is in use.

It is shown that the body 2 is substantially rigid with respect to the one or more inner tubes 3 which, therefore, when inflated protrude from the contact surface 2a so as to press against the user.

The inner tube 3 may be made of polymeric material and in detail of silicone.

The device 1 may comprise at least one circuit 4 for inflating the at least one inner tube 3; and a board 5 for controlling the circuit 4 appropriately in function of said motor 12. The board 5 is configured to command the circuit 4, of the device 1, to inflate the at least one inner tube 3 which, thus inflated, protrudes from the contact surface 2a by pressing against the user and providing feedback to said user.

The board 5 may be configured to control, via the circuit 4, the inflation of the chamber 3 according to the output of said motor 12 appropriately measured by the encoder 13. For example, the board 5 may be configured to control the inflation of the chamber 3 as a function of the number of revolutions and/or the angle of rotation of the output of the motor 12.

Alternatively or additionally, the board 5 may be configured to control the inflation of the chamber 3 as a function of at least one operating parameter of the at least one motor 12. In detail, it is configured to control the inflation of the chamber 3 as a function of said residual current of the at least one motor 12.

Preferably, the board 5 is configured to control the inflation of the at least one chamber 3 according to said residual current of the at least one motor 12 and the output of said motor 12.

The board 5 may comprise an ideal profile, i.e. a profile (e.g. a value) of the current drawn by the prosthesis 10 under ideal conditions. Preferably it comprises an ideal profile associating the current drawn by the motor 12 under ideal conditions with the output of the motor 12. For example, the ideal profile may identify a function of the current drawn as the output varies. In this case, the board 5 may control the inflation according to the residual current given by the difference between the absorbed current and the ideal current with the same output.

The board 5 can be in data connection with the motor 12 so as to detect said functional parameter.

The board 5 may be in data connection with the encoder 13 so as to detect said output measurement.

Preferably, the board 5 may be configured to command the circuit 4 to inflate the chamber 3 according to said at least one functional parameter and the output of said motor 12.

In order to allow data connection between the board and the prosthesis (in detail motor 12 and/or encoder 13), the board 5 may comprise a connector 5a, preferably wired configured to allow said data connection.

The connector 5a may be resolvable so as to allow detachment and/or attachment of the prosthesis 10.

The circuit 4 may be configured to inject and/or extract a fluid into the chamber 3 so as to vary its volume. The inflation fluid may be a gas and suitably air.

It may comprise at least one pump 41 configured to control the inflation of one or more chambers 3 by suitably injecting said fluid into said chamber 3.

The circuit 4 may comprise a pump 41 for each motor 12 of the prosthesis 10.

The circuit 4 may comprise conductors 42 placing in fluid passage connection the pump 41 with the one or more chambers 3.

In the case of a single-actuated prosthesis 10 (i.e., with only one motor 12), circuit 4 may include a pump 41 and conductors 42 ponent in fluid passage connection the pump 41 with the entirety of said at least one chamber 3.

In the case of a bi-actuated prosthesis 10 (i.e. with two motors 12), the circuit 4 may comprise two pumps 41 and pipelines 42 ponent in fluid passage connection each pump 41 with only part of said at least one chamber 3 and, more precisely, each pump 41 with only one chamber 3.

Alternatively, in the case of a bi-actuated prosthesis 10 (i.e., with two motors 12), the circuit 4 may comprise a pump 41; conductors 42 ponent in fluid passage connection the pump 41 with the entirety of said at least one chamber 3; and at least one valve 43 configured to direct the outflow from the pump 41 to one or more of the chambers 3. The valve 43 may be controlled by the board 5 for example depending on the output of said motor 12 and/or at least one operating parameter of said motor 12.

The circuit 4 may comprise a reservoir 44 for storing said fluid.

The conductors 42 may place the tank 44 in fluid passage connection with the pump 41.

The pump 41 is configured to introduce fluid into the one or more chambers 3 (thereby expanding) by drawing it from said reservoir 44.

Pump 41 is configured to move fluid away from said one or more chambers 3 (thereby expanding) by placing it into said tank 44.

The device 1 may comprise control means 6 of the prosthesis 10.

The control means 6 may comprise an EMG sensor.

The means 6 may be integrated into the body 2 and in particular configured to at least partially interpose between the body 2 and the user. They are configured to come into direct contact with the skin so as to receive a command impulse from the user's prosthesis.

The device 1 may comprise a power supply 7 configured to power the device 1 and, in some cases, the prosthesis 10.

The power supply 7 may comprise a battery.

The device 1 may comprise a carter 8 defining a housing volume for at least the board 5, the circuit 4 and if present the power supply 7.

The carter 8 may be constrained, for example solidly, to the body 2.

The carter 8 may be constrained, for example solidly and suitably resolvable, to the rest of the prosthesis 10.

The operation of the prosthetic feedback device 1 described above in structural terms defines a new feedback procedure for prostheses.

The feedback procedure may comprise the prosthetic feedback device 1 and in particular be implemented by the prosthetic feedback device 1.

The feedback procedure may comprise a phase of wearing the prosthesis 10 (e.g. of the forearm and the hand).

In this phase, the user places the body 2 and in detail the contact surface 2a against his own body (for example the amputated end of the arm). This operation places in contact with the user, in detail in correspondence with said amputated end, the one or more chambers 3 and preferably the control means 6.

The feedback procedure may comprise at least one movement phase of the prosthesis 10 in which the motor 12 controls a movement of the prosthesis 10.

In this movement phase, the user, for example by means of said means 6, commands a movement of the prosthesis 10 such as gripping an object. Therefore, the motor 12 is activated by commanding the movement and appropriately the encoder 13 measures the motion output from the motor 12 (such as the number of revolutions of the output shaft).

The feedback procedure may comprise at least one feedback step in which the device 1 provides the user with physical feedback of said movement.

Preferably, the procedure comprises a feedback step for each movement step.

In the feedback phase, the board 5 commands the circuit 4 to inflate one or more chambers 3 according to the motor 12.

In detail, the board 5 can control the inflation of the chamber 3 according to the output of said motor 12 suitably measured by the encoder 13.

Alternatively or additionally, the board 5 may control the inflation of the chamber 3 as a function of at least one operating parameter of the motor 12 and for accuracy of the residual current of the motor 12.

The residual current is determinable as the difference between the current absorbed in the execution of the movement (i.e. in the movement phase) and the ideal current obtainable from the ideal profile.

Preferably, the board 5 commands to the circuit 4 the inflation of the chamber 3 in function of said residual current of the motor 12. In detail, the board 5 commands to the circuit 4 the inflation in function of the residual current given by the difference between the absorbed current and the ideal current with the same output.

The handling and feedback phases can be almost simultaneous so as to have an inflation of one or more chambers 3 simultaneously and, appropriately, proportional to the movement of the prosthesis 10.

Once the movement phase has been completed, The feedback procedure may comprise an additional movement phase in which the prosthesis returns to its initial position and an additional feedback phase in which the board 5 commands the circuit 4 to deflate the chamber 3.

The prosthetic feedback device 1 according to the invention achieves important advantages.

In fact, the prosthetic feedback device 1 allows any prosthesis 10 to provide the user with feedback of the interaction between the prosthesis 10 and the external environment preferably related to a gripping force of an object.

This advantage is particularly relevant in the case of loss due to acquired causes where the device 1 confers the possibility of restoring and in particular making real the latent sensation and therefore the image/processing that remains in the brain relative to the sensation that one had before the loss.

Another advantage is represented by the lightness and ease of use of the device 1 and therefore of the prosthesis 10. In fact, the device 1 is both particularly light and easy to use even immediately by the user.

A further advantage is represented by the fact that the device 1 can also be used with known prostheses without substantially any modification.

The invention is susceptible to variations within the scope of the inventive concept as defined by the claims. Within this scope, all details are substitutable by equivalent elements and the materials, shapes and dimensions can be any.

1. A prosthetic feedback device configured to be associated with a prosthesis; said prosthesis comprises at least one driving motor of said prosthesis; said device comprising
an attachment body configured to come into direct contact with the user and defining a contact surface with said user
at least one air chamber interposed at least partially between said body and said user;
an inflation circuit of said chamber; and
a control board of said device configured to command said circuit to inflate said at least one chamber as a function of said motor so that said at least one inflated chamber protrudes from said contact surface pressing against said user and thus providing said feedback.

2. The prosthetic feedback device according to claim 1, wherein said prosthesis comprises an encoder configured to measure the output of said motor; and wherein said control board is configured to control inflation of said at least one chamber as a function of said output of said motor measured by said encoder.

3. The prosthetic feedback device according to claim 1, wherein said control board is configured to control inflation of said at least one chamber as a function of residual current of said motor.

4. The prosthetic feedback device according to claim 2,
wherein said control board is configured to control inflation of said at least one chamber as a function of residual current of said motor;
wherein said control board comprises an ideal profile associating said residual of said current absorbed by said motor in ideal conditions to said output of said motor; and
wherein said control board is configured to control said inflation of said at least one chamber as a function of said residual current given by a difference between the absorbed current and the ideal current with the same output.

5. The prosthetic feedback device according to claim 1, wherein said at least one air chamber is placed in proximity to the portion of said contact surface proximal to the part of said user wherein the skin deforms during a normal movement of said user.

6. The prosthetic feedback device according to claim 1, comprising a plurality of said at least one chamber.

7. A prosthesis comprise a prosthetic feedback device according to claim 1.

8. The prosthesis according to claim 7 being a hand.

9. A feedback method comprising a prosthesis according to claim 7 and comprising
at least one movement phase of said prosthesis wherein said motor controls a movement of said prosthesis: and
at least one feedback phase wherein said board commands said circuit to inflate said at least one chamber as a function of said motor so that said at least one inflated chamber protrudes from said contact surface pressing against said user and thus providing said feedback.

10. The feedback method according to claim 9, wherein said movement and feedback phases are performed substantially simultaneously so as to have inflation of said at least one chamber simultaneously with said movement of said prosthesis.

11. The prosthetic feedback device according to claim 2, wherein said at least one air chamber is placed in proximity to the portion of said contact surface proximal to the part of said user wherein the skin deforms during a normal movement of said user.

12. The prosthetic feedback device according to claim 1, comprising a plurality of said at least one chamber; and
wherein said circuit comprises a pump, conductor placing said pump in a fluid passage connection with all of said chambers and at least one valve configured to direct the flow at the outlet of the pump towards part of said chambers so as to vary the inflation of said chambers.

13. The feedback method according to claim 9, wherein said prosthesis being a hand.
